(19) United States
(12) Patent Application Publication
(10) Pub. No.: US 2004/0194788 A1
(43) Pub. Date: Oct. 7, 2004

(54) Nose bleed treatment device and method

(76) Inventor: Carl Scott Sweet, Kimberly, ID (US)

Correspondence Address: PEDERSEN & COMPANY, PLLC, P.O. BOX 2666, BOISE, ID, 83701, US

(21) Appl. No.: 10/407,775

(22) Filed: Apr. 2, 2003

(51) Int. Cl. A61F 9/00

(52) U.S. Cl. 128/858; 606/191; 606/201

(57) ABSTRACT

Embodiments of a pressure insert are disclosed for assisting in slowing and stopping nose bleeds. The insert is preferably a firm or non-compressible insert with contours that match or come close to matching the contours of a human's upper gum line and inside surface of the upper lid, which define the buccal cavity. The insert is curved rearward and has generally rounded and smooth edges, and preferably has notches, trough, or contours for conforming over and around the user's frenulum. Preferably, the front surface and the rear surface are generally parallel and both curve around to match, in general, the curvature of the row of front, upper teeth. Alternatively, the insert may be planar prior to use, but may flex to conform to the upper anterior gums after insertion into the buccal cavity.

### BACKGROUND OF THE INVENTION

1. Field of the Invention

The invention relates, in general, to apparatus and methods for treatment or prevention of nose bleeds in humans. More specifically, the invention relates to a device for slowing or stopping nose bleeds without medicines, operations, or contact with the nasal passages themselves. Specifically, the invention relates to a method of applying pressure under the upper lip of a person experiencing a nose bleed to slow or stop a nose bleed, and a simple and effective device for performing the method.

2. Related Art

Through the years, many people have developed home remedies for combating the troublesome problem of nose bleeds, which are also called “epistaxis” and “nasal hemorrhaging”.

Nose bleeds tend to trouble some individuals much more than others, and may occur after stress or exercise, or due to dry air conditions or other irritations of the nasal interior tissues. Traditionally, sufferers of nose bleeds tilt their heads back, apply cold compresses to the nose, and/or simply lie still to wait for the clotting that is necessary to stop the bleeding.

Many devices have been developed to assist in stopping nose bleeds. For example, Kern, et al. (U.S. Pat. No. 4,457,756) discloses a tong-shaped clip with absorbent ends for insertion into the nostrils for pressuring the mucosa. Payton (U.S. Pat. No. 4,338,941) discloses an apparatus for arresting posterior nosebleeds, wherein an inflatable bag is inserted into the nasal cavity for inflation by a fluid conveyed into the bag by tubes. Berry (U.S. Pat. No. 4,820,266) discloses a method of stopping nose bleeds that involves inserting a flattened strip of material into the nose, and then pressing the outside of the nose between the thumb and fingers, so that the strip is compressed against the inside surface of the nasal cavity in an area called “Littles Area.” Stangerup (U.S. Pat. No. 5,546,964) discloses a method of treating a nose bleed with a warm water rinse, by using a catheter having a rinsing outlet and an inflatable balloon beyond the rinsing outlet. Knott, et al. discloses an external clip and methods of using an external clip for external nasal compression for treating anterior nosebleeds. Brennan (U.S. Pat. No. 5,011,474) discloses a method of applying pressure to the inside surfaces of the nasal cavity by means of a tampon and sealing cuff combination. Walker (U.S. Pat. No. 5,383,891) discloses a nose bleed kit including an oval-shaped tampon for insertion into the nasal cavity, which expands upon contact with an aqueous fluid.

In general, prior art nose bleed treatment devices for laymen include compression devices for the outside of the nose or pressure devices for the inside surface of the nasal cavity. These devices give mixed results, and those that involve contact with the inside of the nasal cavity are unpleasant or worrisome for many individuals.

Therefore, there is still a need for a simple and effective nose bleed treatment that a person may perform in his/her home without a doctor or nurses' help. There is still a need for a more pleasant and reliable treatment device that does not involve inserting any object into the nose. The present invention meets these needs.

### SUMMARY OF THE INVENTION

The present invention comprises a device for slowing or stopping nose bleeds that is inserted under the upper lip of the person experiencing the nose bleed. The invented method comprises applying pressure to the tissues inside the mouth near the nose to slow or stop bleeding from the nasal tissues. Preferably, the invented device and method involve an insert device that is placed in the space between the inner surface of the upper lip and the teeth and gum area, which is called the buccal cavity. The preferred device is adapted to be large enough and firm enough that it fills a significant amount of the buccal cavity volume in front of the anterior teeth and causes pressure substantially all along the surfaces it contacts, as the upper lip tends to compress the device against the upper gum and adjacent areas.

The preferred device is an elongated pressure member, which is firm but slightly-conforming and/or flexible, so that, when inserted under the upper lip, the pressure member is generally comfortable to the user but still firm enough to cause significant pressure on the inner surface of the upper lip, the upper region of the upper buccal cavity, and on the outer, upper gum surfaces.

It is an object of the invention to provide an economical and easily-used device that may assist nose bleed sufferers to treat their own nose bleeds. It is an object of the present invention to provide a device that is easily packaged in a clean and/or antiseptic package and that is easily-carried for quick use when needed. It is an object of the present invention to provide a treatment device and method that preferably do not comprise applying medicines, drugs, or other chemicals or treatments to the body other than the mechanical/physical effect of the properly-placed pressure in the buccal area. It is an object of the present invention to provide a device that does not contact the nose or nasal passages and does not involve insertion of any object or material on or inside the nose. It is another object to provide a disposable and economical device that may be purchased without prescription by individuals and/or handed out by school nurses, by day-care providers, or other aides, without medical training and without substantial training.

### BRIEF DESCRIPTION OF THE DRAWINGS

FIG. 1 portrays the anatomy of the mouth, showing the buccal cavity wherein the invented device is placed.

FIG. 2 is a front view of a user, with one embodiment of the invented device in dashed lines in place under the upper lip of the user.

FIG. 3 is a front view of one embodiment of the invented pressure insert.

FIG. 4 is a back view of the embodiment of FIG. 3.

FIG. 5 is a top view of the embodiment of FIGS. 3 and 4.

FIG. 6 is a bottom view of the embodiment of FIGS. 3-5.

FIG. 7 is a front view of the embodiment of FIGS. 3-5, showing the locations for the cross-sections taken for FIGS. 7a and 7b.

FIG. 7a is a cross-section taken along the line 7a-7a in FIG. 7.

FIG. 7b is a cross-section taken along the line 7b-7b in FIG. 7.

FIG. 8 is a front view of a user, with an alternative embodiment of the invented device in dashed lines in place under the upper lip of the user.

FIG. 9 is a front view of the alternative embodiment of the invented pressure insert of FIG. 8.

FIG. 10 is a rear view of the embodiment of FIGS. 8 and 9.

FIG. 11 is a top view of the embodiment of FIGS. 8-10.

FIG. 12 is a bottom view of the embodiment of FIGS. 8-11.

FIG. 13 is an end view of the embodiment of FIGS. 8-12.

FIG. 14 is a cross-sectional view of the embodiment of FIGS. 8-13, viewed along the line 14-14 in FIG. 9.

FIG. 15 is a cross-sectional view of an alternate embodiment of the invented insert, having a rearwardly-curved firm core surrounding by a cushioning cover.

### DESCRIPTION OF THE PREFERRED EMBODIMENTS

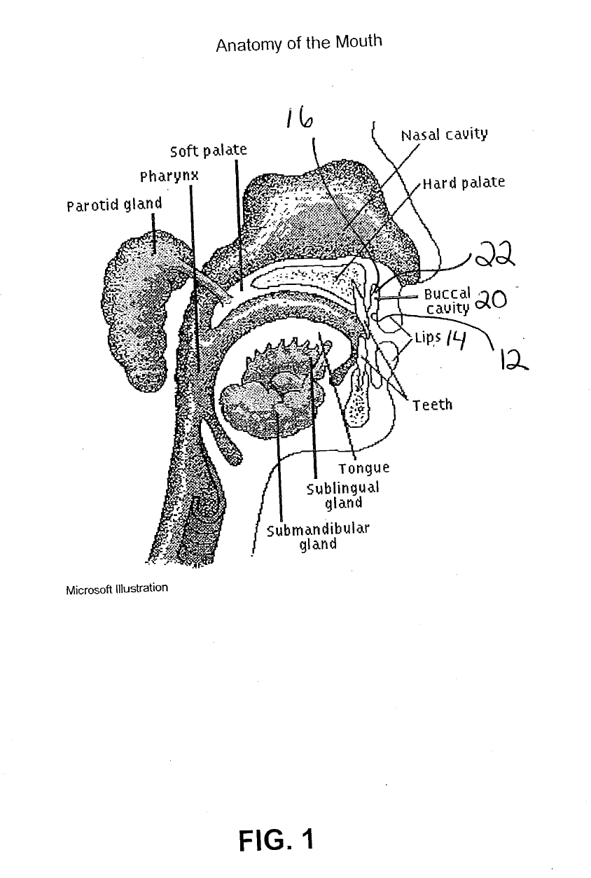

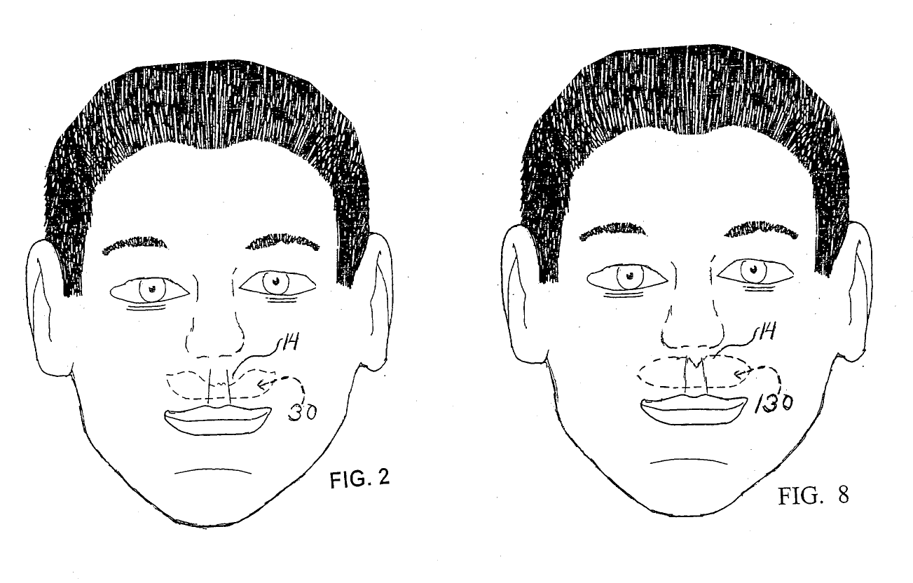

Referring to the Figures, there are shown some, but not the only, embodiments of the invented device and methods of using them. FIG. 1 illustrates the human anatomy of the mouth, wherein there is a space between the inner surface 12 (rear surface) of the upper lip 14 and the front gum surface 16 of the upper anterior gums, which space is called the “buccal cavity 20.” This cavity 20, therefore, is generally defined by the tissue (inner surface 12) curving up, around about 180 degrees at the upper portion 22 of the cavity, and down along the gum surface 16. This arched buccal cavity area, therefore, is defined at its front wall by the lip, at its rear wall by the gums, at its top by the transition between the lip, and by its open bottom end.

The preferred pressure insert 30, according to one embodiment of the invention, is inserted into the buccal cavity 20, so that it preferably fills a large portion of the buccal cavity at the anterior of the mouth, and fits well up into the upper portion 22 of the cavity. This placement of the pressure insert 30 is done by lifting the upper lip out from the teeth by one's own lip and mouth 30 movement, or, more preferably, by grasping the upper lip and pulling it out to make room for the pressure insert 30. The pressure insert 30 is gently pushed up as far as comfortable into the buccal cavity 20, and the lip is then lowered down on the insert 30 so that the natural pressure of the lip against the pressure insert and the tightness of the fit of the insert 30 in the cavity 20 cause significant pressure on the skin and tissues being directly contacted by the pressure insert 30, and, consequently, significant pressure on the tissues in the adjacent areas of the mouth.

The inventor believes that this pressure against the surfaces of the buccal cavity transmits pressure to the blood vessels inside the tissue and possibly to other adjacent areas of the mouth and nose/nasal region, which slightly constricts flow of blood in and/or to areas of the mouth and nose/nasal regions. This has the effect of slowing or stopping many nose bleeds. While the inventor does not currently have a specific theory about which blood vessels are constricted by the invented device, he knows that the invented device is effective in stopping many different types and locations of nose bleeds for many people, and that the device may be easily and safely used by a person without medical training or knowledge.

Preferably, the pressure insert 30 is an elongated member having a central area 32 and two ends, which may be called a right end 34 (for placement toward the user's right) and a left end 36 (for placement toward the user's left). The insert, in general, is preferably either pre-formed, or flexed during use, to fit the curve of the anterior upper gum line, to curve generally smoothly under the lip, to present generally smooth surfaces to the upper gums and lip. This way, the insert 30 does not force large or painful shapes underneath the lip, and is unlikely to hurt or damage the tissue of the mouth. As illustrated in FIGS. 5 and 6, the insert 30 curves generally rearward, for extending along and above the gum line. The curvature should match a typical person's anterior mouth shape. For example, a curvature of about a 3-4 inch radius may be appropriate for an adult or about a 2-3 inch radius may be appropriate for a child.

Inserts that are “pre-formed” into a preferred rearward curvature, are formed/shaped at the time of manufacture by means of the insert being molded, cut or otherwise formed/shaped into the desired shape. Alternatively, inserts that are not pre-formed to curve rearward are preferably planar prior to insertion into the mouth, but are also preferably flexible enough to bend into the preferred gum-line curvature upon insertion and compression under (behind) the upper lip.

Some embodiments of the invented insert may be “pre-formed” to curve upward on either side of the central area, by means of molding, cutting or other forming/shaping during manufacture. Such upward curving of the ends forms a trough in the top edge, for extending up around the frenulum and allowing the insert to reach well up into the buccal cavity. In other embodiments, a notch may be supplied at the center of the top edge to specifically provide room for and to receive the frenulum, in addition to or instead of a trough.

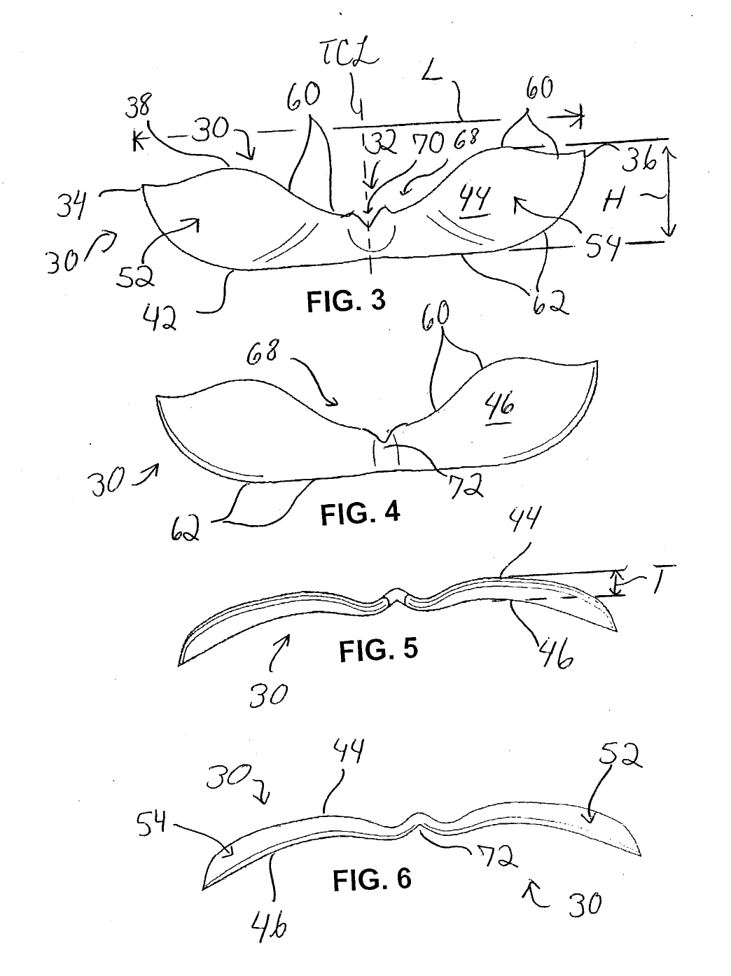

The insert 30 shown in FIGS. 3-6 is generally an elongated member with pre-formed rearward curvature that allows it to comfortably fit around the curve of the top anterior gum line. The length of the insert 30 is its largest dimension, followed by its height, with its thickness being small relatively to both the length and height.

When viewed from the front, one may see that end portions 52, 54 of insert 30 in FIG. 3-6 are taller than the central area 32, by means of the top edge 60 being curved to define pre-formed or pre-cut trough 68 and notch 70. The top edge 60 curves gradually downward toward the central area 32 to form trough 68 and, at the transverse centerline, the top edge 60 curves more sharply downward to form v-shaped notch 70. The trough and notch are both intended to to avoid the frenulum and to allow the end portions 52, 54 to reach as far as possible and comfortable upward on either side of the frenulum and up into the upper portion 22 of the buccal cavity.

In the embodiment of FIGS. 3-6 and in many embodiments, the bottom edge 62 of the insert 30 is generally straight near the central area and curves upward toward each end to form the curved end portions 52, 54. The bottom edge of the end portions 52, 54 may be generally curved upwards all the way to the top of the insert, so that the end extremities are at or near the top edge 60 of the insert 30. Alternatively, for example, the bottom edge may meet the top edge midway up the insert, so the end extremities are near the insert's longitudinal center line, as in FIG. 8. Other right and left end shapes may also be used.

Many sizes of insert may work well, for different sizes and ages of individuals. The insert 30 is preferably 1-3.0 inches long from end 34 to end 36, and about 0.33-1.0 inch wide from top 38 to bottom 42, and about ⅛-½ inches thick from front side 44 to rear side 46.

A preferred adult size has a length (L) from end 34 to end 36, and a height (H) from top 38 to bottom 42, and a thickness (T) from front side 44 to rear side 46. The preferred adult size insert 30 thickness is about ½ of the height. A preferred adult insert may be about 2.5 inches long, about ½-¾ inches wide and about ¼ inch thick. The combination trough 68 with notch 70 in the insert 30 of FIGS. 3-6, wherein the combination extends down into the central area a distance equal to about ½ of the height.

A kit of several sizes of inserts may be provided, so that both men, women, and children may have inserts that comfortably fit their mouths. Or, an adult size may be provided, with instructions to cut down the outer perimeter of the insert for use by children. A preferred child's size would be, for example, about 1.5 inches long, about ⅓-½ inch tall, and about ⅛-¼ inch thick.

As a further accommodation to a user's buccal cavity shape and to the frenulum and surrounding tissue surface, the rear surface 46 of the insert may further include an optional channel 72. This channel 72 extends transversely from the top edge to a position at or close to the bottom edge, and provides additional concave space for ensuring that the rear surface 46 will not hurt or damage the frenulum and adjacent tissue.

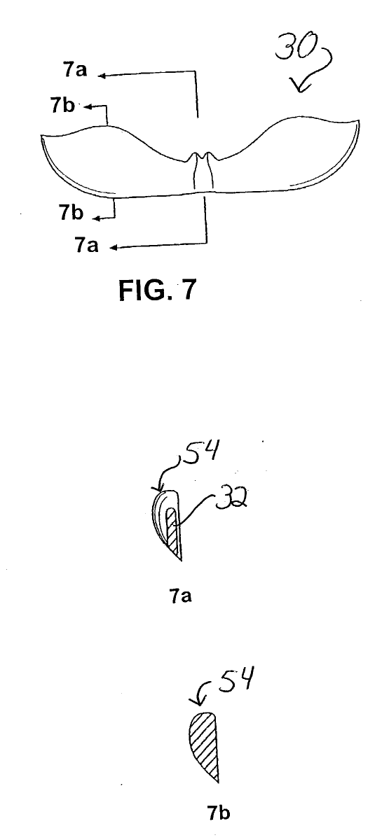

FIGS. 5 and 6 illustrate that the right and left end portions 52, 54 may be thicker than the central area 32, for filling the space and increasing the pressure against the skin in the end portions 52, 54 respective areas of the buccal cavity. FIGS. 7, 7a, and 7b further illustrates insert 30 to best advantage, by showing cross-sections that reveal that the ends of the insert are bulged out and have larger cross-sectional areas that the center area 32.

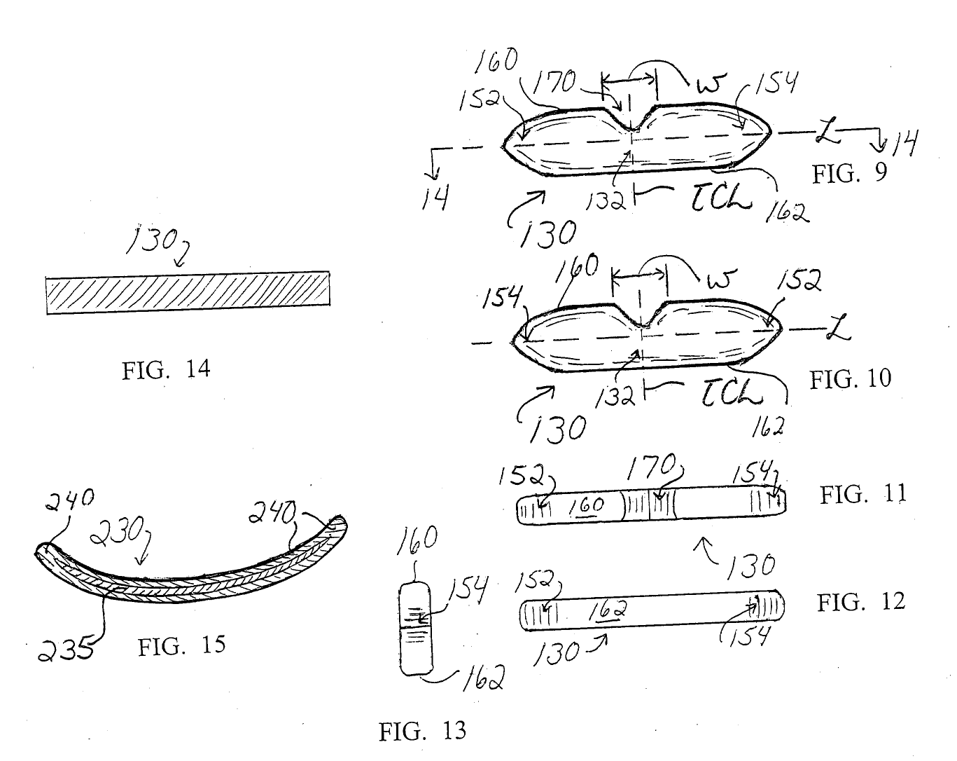

Alternative versions of the invented insert may fit and be effective for other wearers. For example, an alternative insert 130, as shown in FIGS. 8-11, is a plate structure that has planar front and rear surfaces that are substantially and preferably parallel to each other. Insert 130 features less curvature of its edges and surfaces and less variation in height and thickness compared to insert 30. For example, the central area 132 of insert 130 is about the same thickness as the end portions 152, 154, and the insert 130 is substantially the same thickness all along its length and height. Further, insert 130 features a top edge 160 and a bottom edge 162 that are substantially straight, except for the single notch 170 sharply extending into the body of the insert in the top edge 160 near the transverse centerline (TCL). The ends 152, 154 may be rounded or otherwise shaped for comfort.

Examples of dimensions for the insert 130 in FIGS. 8-14 are about 2.5 inches long (preferably 2.25-2.75 inches), about ½ inch tall (preferably ⅓-⅔ inches tall), and about ¼ inch thick (preferably ⅙-⅓ inches). Preferably, the thickness of the insert is about ½ of the height. The notch 170 width (W) is about ⅕ of the total length of the insert 130, for example, about ½ inch wide for a 2.5 inch long insert. The notch 170 extends down into the insert a distance equal to about ⅓ of the height, for example, for a ½ inch high (tall) insert, the preferred notch is about 3/16 inch deep. A frenulum notch for a child would preferably be about ⅓-½ inches across and about ⅛-3/16 inch deep.

Preferably, the invented insert 30, 130 (and 230 in FIG. 15) fits above the central 4-6 anterior teeth, that is, the central incisors, the lateral incisors, and, optionally, also the canines. Alternatively, and less preferably, the insert 30, 130, 230 may be effective on one side of the other of the frenulum, rather than centered across the front of the mouth, and, hence, some embodiments may not require a trough or notch.

The insert may be made of various materials, which are adapted to be clean for use in the mouth, non-splintering, break-resistant, and slightly flexible and slightly cushioning. For example, the inventor prefers silicone, latex, polyfoam, or even paper or cardboard compositions preferably coated. Various plastics may be used, and various layered or filled compositions, for example, a shell containing a cooled liquid. The preferred insert 30, however, does not contain any cooling or chilling system or material. The insert may be made of several layers, parts, or sections connected together, but, in its simplest structure, it is a one-piece, solid, firm but non-abrasive and non-gouging insert that causes pressure in the buccal cavity. In its simplest structure, it has continuous surfaces rather than having holes or apertures or significant texture. Preferably, the insert is not adsorbent and is preferably liquid-resistant, does not harbor bacteria, and is washable, for reuse. Alternatively, the insert may be disposable.

Inserts according to the invention are preferably non-compressible or slightly compressible, so that an insert made with certain dimensions will substantially maintain those dimensions where inserted in the mouth and pressured by the upper lid. Compression may be acceptable in some embodiments, but this would require a larger insert to provide the desired pressure on the buccal cavity surfaces, makes the performance of the insert less predictable, and makes the insert less conveniently installed into the mouth.

While a solid insert is preferred as shown in the cross-sectional view in FIG. 14, the invented insert and method may comprise alternative compositions. For example, as illustrated in FIG. 15, the insert 230 may be made of a firm or rigid core 235, covered with a flexible or cushioning substance 240 that provides pressure but is not painful or uncomfortable.

While various shapes may be used, the preferred insert 30 applies substantial pressure in the upper portion of the buccal cavity and especially in the area above the teeth. As discussed above, the insert may be produced in various sizes, and/or with a trim-to-fit feature, to fit various people's mouths. Alternatively, the invented insert may be custom-made to fit a particular person's mouth. The thickness of the insert and bulging of the insert, if any, designed to fill and cause pressure in a 1-3 inch long portion of the buccal cavity, should not be trimmed away to the extent that reduces the compressive pressure inside the buccal cavity, and, hence, reduces the effectiveness of the insert.

An insert according to the invention may also be made by molding an insert to the contours of a particular user's buccal cavity. Such an insert may be made for a user that has particular problems with comfort, or that has particular problems with nose bleeds that require a custom fit.

The invented method and device preferably provide constant pressure for several minutes up to about ½ hour, to safely slow and stop nose bleeds. Even if the user needs to use it for extended periods of time or repeatedly, the invented insert is not so large as to cause a serious protruding of the upper lip, and, hence, may be generally concealed under the upper lip. This way, the invented insert is a non-embarrassing method for reducing bleeding and the mess caused by bleeding of the nose.

Although this invention has been described above with reference to particular means, materials and embodiments, it is to be understood that the invention is not limited to these disclosed particulars, but extends instead to all equivalents within the scope of the following claims.

I claim:

1. A nose bleed treatment device comprising:
an insert for placement in a user's mouth for assisting in stopping nose bleeds, the insert comprising an elongated and non-absorbent member adapted to apply pressure in the user's buccal cavity.

2. The device as in claim 1 having a top edge, bottom edge, left end and right end, the top edge having a notch extending into the insert generally midway between the left end and right end.

3. The device as in claim 1 having a top edge, bottom edge, left end and right end, the top edge curving to form a trough generally midway between the left end and right end.

4. The device as in claim 3 having a v-shaped notch extending into the insert at a transverse centerline midway between the left end and right end.

5. The device as in claim 1 wherein the insert is non-compressible so that it is not compressed when pressed by the user's lip in the buccal cavity.

6. The device as in claim 1 wherein the insert is flexible so that the insert curves rearward to rest along the upper gum line at the buccal cavity.

7. The device as in claim 1 wherein the insert has a front surface and a rear surface, and the insert is curved rearward so that the front surface is convex and the rear surface is concave.

8. The device as in claim 1, wherein the insert has a right end, a left end, a length between said right and left end, a top edge, a bottom edge, a height between said top edge and bottom edge, a front surface, a rear surface, and a thickness between said front surface and rear surface, wherein said length is 2.25-2.75 inches, said height is ⅓-⅔ inches, and said thickness is ⅙-⅓ inches.

9. The device as in claim 7, wherein said thickness is substantially constant all along the length of the insert.

10. The device as in claim 1, wherein the insert has a length between two ends, a height between a top edge and a bottom edge, and a thickness between a front surface and a rear surface, and wherein the insert near the top edge has a thickness greater than about twice the thickness near the bottom edge.

11. The device of claim 1, wherein the insert has a central area, a length between two ends, a height between a top edge and a bottom edge, and a thickness between a front surface and a rear surface, wherein the insert near each of the two ends has a thickness greater than about 1.5 times the thickness near the central area.

12. The treatment device of claim 1, wherein the insert is curved on a radius in the range of 3-4 inches for an adult's use.

13. The treatment device of claim 1 wherein the insert is curved on a radius in the range of 2-3 inches for a child's use.

14. A nose bleed treatment device comprising:
an insert for placement in a user's mouth for assisting in stopping nose bleeds, the insert comprising an elongated member adapted to apply pressure in the user's buccal cavity;
the insert having a central area, a length between two ends, a height between a top edge and a bottom edge, a thickness between a front surface and a rear surface, and a transverse centerline midway between the two ends and perpendicular to the length, wherein the insert has a notch in said top edge at said transverse centerline, said notch being for receiving and extending around frenulum tissue of the user.

15. The device of claim 14, wherein the insert is a plate of non-compressible material adapted to curve along the user's upper gum line by means of being curved.

16. The device of claim 14, wherein the insert is a plate of material adapted to curve along the user's upper gum line by means of said material being flexible.

17. The device of claim 14, wherein the insert is a plate of rigid material with a cushioning cover.

18. A method of stopping a nose bleed comprising placing an insert under the upper lip of the user, wherein the insert is adapted to be a size and shape that causes pressure against the tissue of the user in the upper buccal area, and, thereby, constricting blood vessels and slowing blood flow from a nose bleed.
